(19) United States
(12) Patent Application Publication
(10) Pub. No.: US 2015/0133824 A1
(43) Pub. Date: May 14, 2015

(54) TRAIN-OF-FOUR MEASURING CLIP

(71) Applicant: Rutgers, The State University of New Jersey, New Brunswick, NJ (US)

(72) Inventors: Glen Atlas, Livingston, NJ (US); Edan Golomb, Hoboken, NJ (US); Kyle Ripley, Weymouth, MA (US); Derek Smith, Boise, ID (US); Jennifer Trinh, Jersey City, NJ (US); Vikki Hazelwood, Wayne, NJ (US)

(73) Assignee: Stevens Institute of Technology, Hoboken, NJ (US)

(21) Appl. No.: 14/538,384

(22) Filed: Nov. 11, 2014

Related U.S. Application Data
(60) Provisional application No. 61/902,621, filed on Nov. 11, 2013.

(51) Int. Cl. A61B 5/11

(52) U.S. Cl. CPC A61B 5/1106
USPC 600/595

(57) ABSTRACT

A monitoring apparatus includes a clip assembly configured to be secured on a patient's digit. The clip assembly houses an accelerometer which measures acceleration in three dimensions. A microcontroller is configured to receive acceleration data from the accelerometer representative of the acceleration measured in each of the three dimensions. A processor is configured to analyze the acceleration data and determine and output a train-of-four ratio based the acceleration measured in each of the three dimensions.

This application claims the benefit of U.S. Provisional Application No. 61/902,621 filed on Nov. 11, 2013, the contents of which are incorporated herein by reference.

### FIELD OF THE INVENTION

This invention relates to neuromuscular blockade monitoring. More particularly, the invention relates to a device and method for monitoring neuromuscular blockade.

### BACKGROUND OF THE INVENTION

In surgery requiring general anesthesia, doctors use anesthetics to render the patients unconscious and then administer neuromuscular blocking agents (NMBAs) to relax the muscles and allow for easy tracheal intubation. Because NMBAs paralyze all muscles including the diaphragm, patients cannot breathe independently. It is therefore critical to ensure that neuromuscular blockade (NMB) has been eliminated prior to tracheal extubation.

The Train-of-Four (TOF) ratio is the standard that has been developed to monitor NMB. The ulnar nerve is stimulated with electrical current four times, eliciting four thumb twitches. TOF monitoring measures the contraction of the adductor pollicies by electrically stimulating the ulnar nerve. The ratio of the force of the fourth twitch to the force of the first is known as the TOF ratio.

Monitoring the level of neuromuscular blockade (NMB) throughout surgery is critical in ensuring patient safety, especially in preparing for extubation. Premature extubation is a life-threatening complication that results in expensive stays in the intensive care unit.

Anesthesiologists need an inexpensive, practical device that works with their existing systems to objectively determine the appropriate time to extubate patients.

### SUMMARY OF THE INVENTION

Currently, the products on the market consist of two interfaced components: nerve stimulator and muscle response sensor. Current devices on the market typically either only provide stimulation or provide both stimulation and measurement of the thumb twitch force as part of a multi-component system. As many anesthesiologists already have a nerve stimulator, the purchase of current products renders their existing stimulators useless. The present invention provides an independent muscle response sensor that works with existing nerve stimulators to accurately measure NMB without receiving input from the nerve stimulator.

An exemplary TOF clip in accordance with at least one embodiment of the invention is used to improve the standard evaluation method of monitoring the level of NMB in surgery patients under anesthesia. The standard process is a two-step process. The first step is electrical stimulation of the ulnar nerve via a peripheral nerve stimulator and the second step is the measurement of the resulting thumb twitch force either by an objective or subjective method. The stand-alone TOF clip will improve the standard process by providing an objective method to monitor thumb twitch force when using an existing independent stimulator.

Existing objective neuromuscular monitoring systems typically consist of a nerve stimulator interfaced with a muscle response sensor. Because the stimulator and sensor are interfaced, the systems can easily detect the difference between noise generated from unwanted thumb movement and the muscle response from the nerve stimulation. The sensor in these systems starts recording when the nerve is stimulated thereby eliminating potential error due to noise before stimulation.

In an exemplary embodiment, the TOF clip of the present invention is an independent muscle response sensor, meaning that it is not interfaced with a stimulator. The challenge with this is that the device must be able to differentiate between unwanted thumb motion and the thumb twitches caused by nerve stimulation. The exemplary TOF clip incorporates a unique algorithm to differentiate between these motions. The algorithm identifies the correct thumb acceleration peaks resulting from nerve stimulation by searching the data for peaks at the same frequency that the stimulation occurs.

The algorithm also ensures gravity will not alter thumb twitch acceleration readings. By combining the acceleration readings from all three of the accelerometer axes, the algorithm is able to remove gravity's effect on the acceleration data. Another potential benefit of the algorithm is that it can be used such that the TOF clip generates accurate data even if it is placed on the thumb angled in a rotated manner.

In at least one aspect, the present invention provides a thumb clip containing a three-axis accelerometer that measures acceleration. When the thumb twitches in response to nerve stimulation, the acceleration data is collected via a microcontroller, graphed and the standard ratio used in monitoring neuromuscular blockade is displayed.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an exploded isometric view of an exemplary embodiment of a finger gripping housing in accordance with the invention.

FIG. 2 is a top plan view of an exemplary TOF clip with a portion of the shell member removed to show the accelerometer.

FIG. 3 is a side elevation view of an exemplary TOF clip.

FIG. 4 is a perspective view illustrating the TOF clip attached to a microcontroller.

FIG. 5 is a schematic view illustrating the accelerometer attached to the microcontroller.

FIGS. 6 and 7 are flow charts illustrating an exemplary procedure for determining the TOF from the observed accelerometer data.

FIG. 8 is a graph illustrating observed thumb acceleration in three axes.

FIG. 9 is a graph illustrating compiled thumb acceleration in three axes.

FIG. 10 is a graph illustrating analyzed thumb acceleration.

FIG. 11 is a bar graph of observed thumb acceleration.

FIG. 12 is a bar graph of hand motion frequency ranges.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

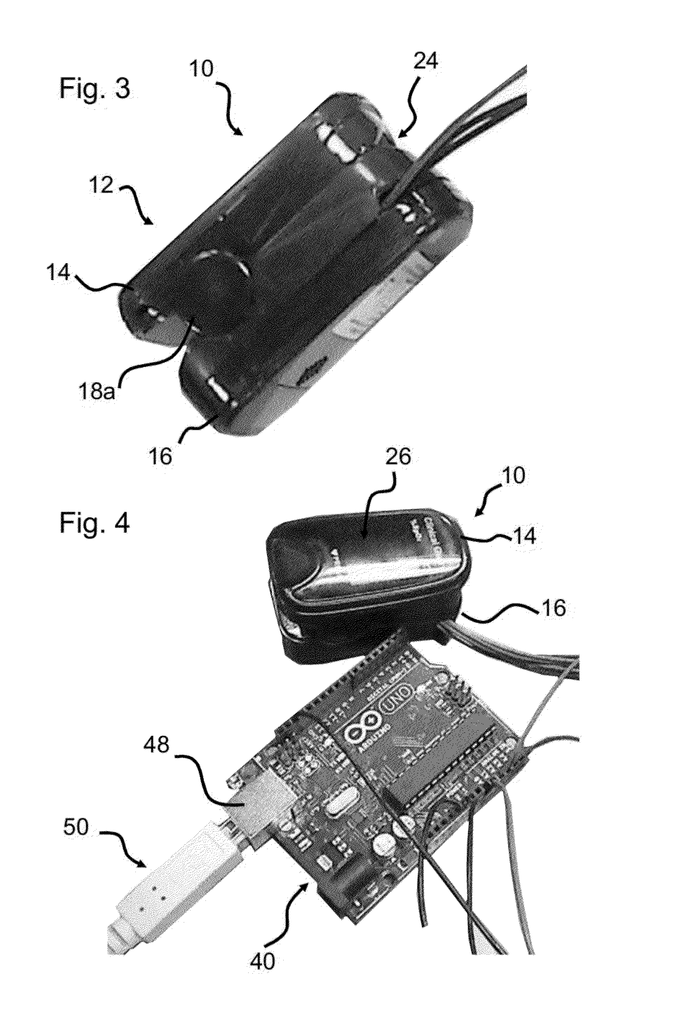

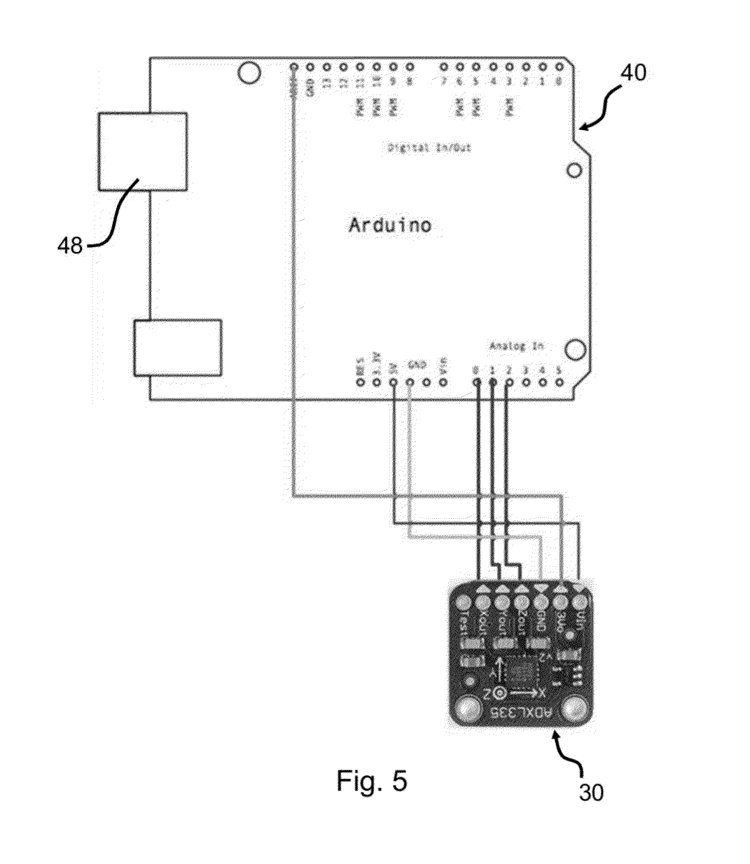

Referring to FIGS. 1-5, an exemplary embodiment of a TOF clip 10 in accordance with an embodiment of the invention will be described. The exemplary TOF clip 10 includes a finger gripping housing 12 defined by opposed shell members 14 and 16. In exemplary embodiment, the shell members 14 and 16 are manufactured from an acrylonitrile butadiene styrene (ABS), however, other materials may be utilized. The two shell members 14, 16 are connected by a hinge 18a, 18b and are biased to a finger gripping position (see FIG. 3) by one or more springs 20 or the like. A respective interchangeable grip plate 22 may be positioned in each shell member 14, 16 as illustrated in FIG. 1. Once assembled, there is an opening 24 on the side farther from the base of the hinge 18a, 18b that allows a patient's digit, thumb, toe, or the like, to be inserted between the two shell members 14, 16 and is secured between them. The finger gripping housing 10 may include an outlet display 26 positioned in one of the shell members 14 and configured to display a calculated TOF. The finger gripping housing 10 may be made via an injection molding process. The finger gripping housing 12 may be configured from a finger pulse oximeter. The TOF clip 10 can be made compatible for pediatric, adult and geriatric patients.

A Thumb Clip Slip Test was conducted to determine if a single set of springs 20 of the modified clip was sufficient enough to prevent the thumb clip 10 from slipping during a thumb twitch. It was found that one pair of springs 20 was sufficient (P<0.0001) to hold the clip in place. Data of the test is provided in Table I below.

TABLE 1

| | Subject | | | | | | | | | |
|---|---|---|---|---|---|---|---|---|---|---|
| | 1 | | 2 | | 3 | | 4 | | 5 | |
| | No. of Springs | | | | | | | | | |
| | 1 | 2 | 1 | 2 | 1 | 2 | 1 | 2 | 1 | 2 |
| Clip | 1 | 0 | 0 | 1 | 0 | 0 | 0 | 0 | 6 | 0 |
| Slip | 0 | 1 | 0 | 0 | 1 | 1 | 1 | 0 | 0 | 0 |
| Distance | 0.5 | 1 | 0 | 0 | 0 | 0 | 1 | 0 | 0 | 0 |
| (mm) | 1 | 0 | 0 | 0.5 | 1 | 1 | 0 | 0.5 | 1 | 0 |
| | 2 | 0 | 1 | 0.5 | 0 | 1 | 3 | 0 | 2 | 0 |
| | 1 | 0.5 | 0 | 1 | 0 | 1.5 | 2 | 0 | 0 | 0 |

The following provides data analysis for one set of springs:

One-Sample z-Test/Upper-Tailed Test:

95% confidence interval on the mean:
] 0.437, +Inf [

| | |
|---|---|
| Difference | 2.817 |
| z (Observed value) | 12.217 |
| z (Critical value) | 1.645 |
| p-value (one-tailed) | <0.0001 |
| alpha | 0.05 |

One-Sample z-Test/Lower-Tailed Test:

95% confidence interval on the mean:
] −Inf, 1.196 [

| | |
|---|---|
| Difference | −1.183 |
| z (Observed value) | −5.133 |
| z (Critical value) | −1.645 |
| p-value (one-tailed) | <0.0001 |
| alpha | 0.05 |

It can be concluded that the clip did not move when one set of springs was used with no batteries. Lack of clip slippage was essential to calculating the proper TOF ratio because slippage changes the moment arm of rotation and would thus affect the acceleration data causing inaccurate readings. While one set of springs was determined suitable, other configurations including more or fewer springs or other biasing elements may be utilized.

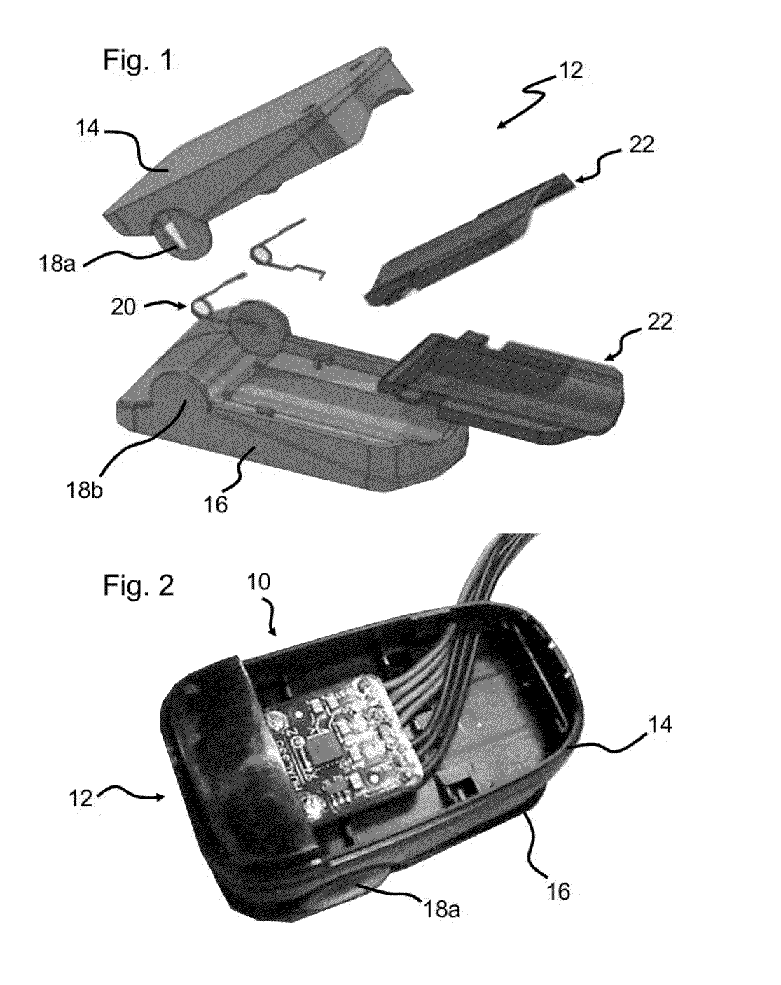

Referring to FIGS. 2 and 4-5, an accelerometer 30 is secured within the shell member 14 with wires 32 extending from the shell member 14 to a microcontroller 40. While wires are shown in the illustrated embodiment, wireless communication or other connectivity may be utilized. In an exemplary embodiment, the accelerometer 30 is a three-axis mems accelerometer manufactured by AcceleroMetrix™ and the microcontroller 40 is an Arduino microcontroller. Other accelerometers and microcontrollers may be utilized. In the exemplary embodiment, the connection of the accelerometer 30 with the microcontroller 40 provides power to the TOF clip 10. The circuitry is preferably configured to filter out undesired frequencies. The filtered signal is then digitized by a data acquisition device (DAQ) within the microcontroller 40. The microcontroller 40 has an outlet 48 which connects to a computer connection cable 50 which connects to one or more of a computer, monitor, display or the like. Software within the microcontroller 40 or an external device, e.g. computer, is utilized to analyze the digitized accelerometer data. The analyzed data is then presented to the user on the display 26 of the TOF clip 10 and/or on an external display. The data may be transferred to an external display via a wired or wireless transmission, for example. Bluetooth™ transmission, to a monitor, computer screen, tablet, smartphone or the like. The transmission may be a two-way transmission such that other information, for example, advertising materials, hospital protocols, etc., may be displayed on the display 26 when the analyzed data is not being presented.

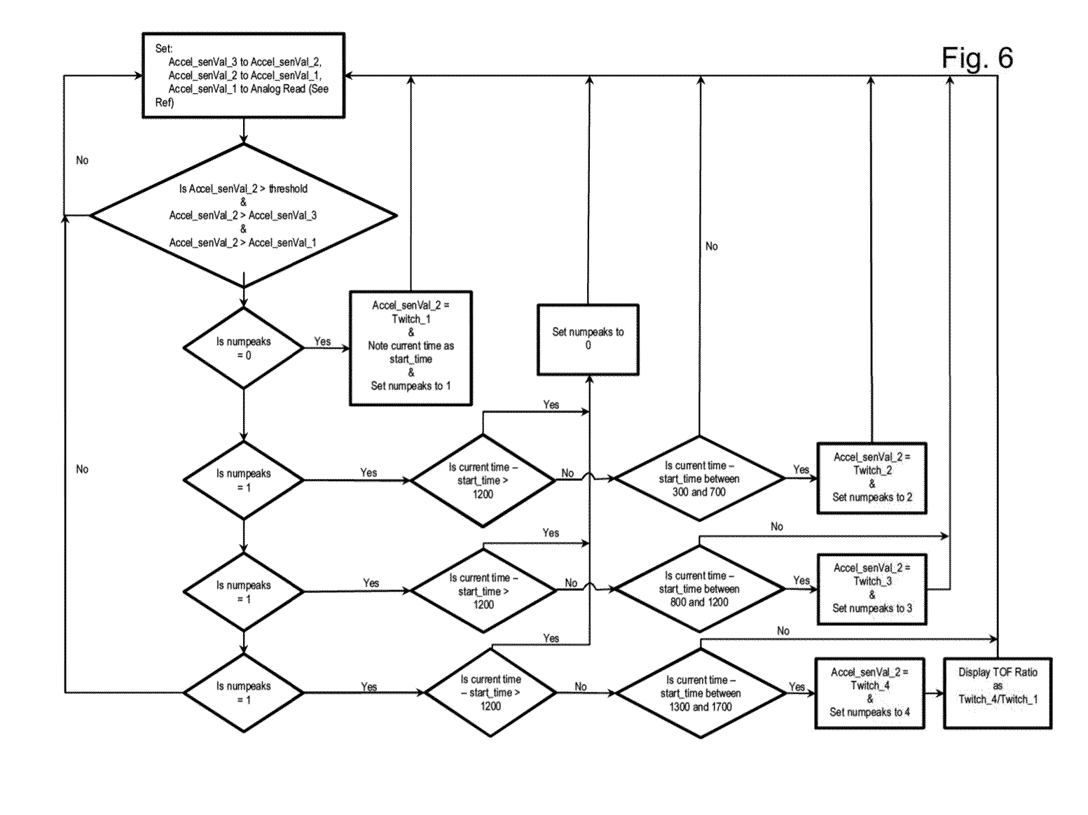

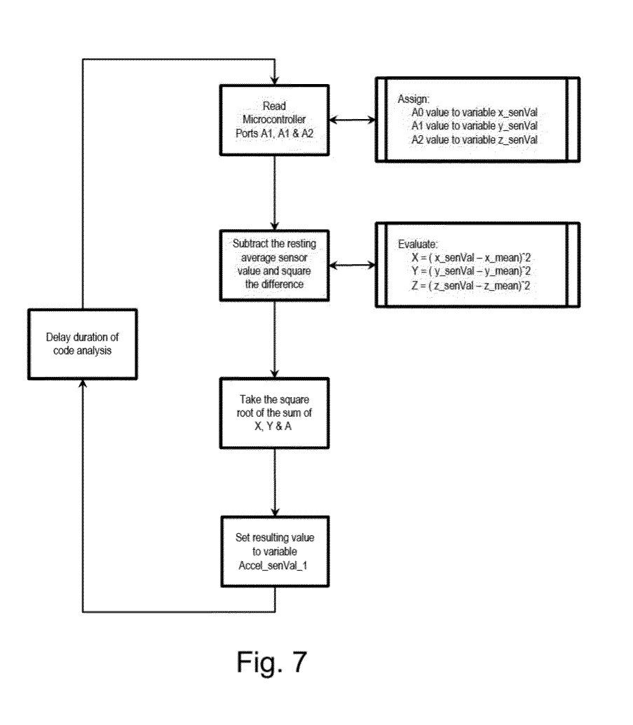

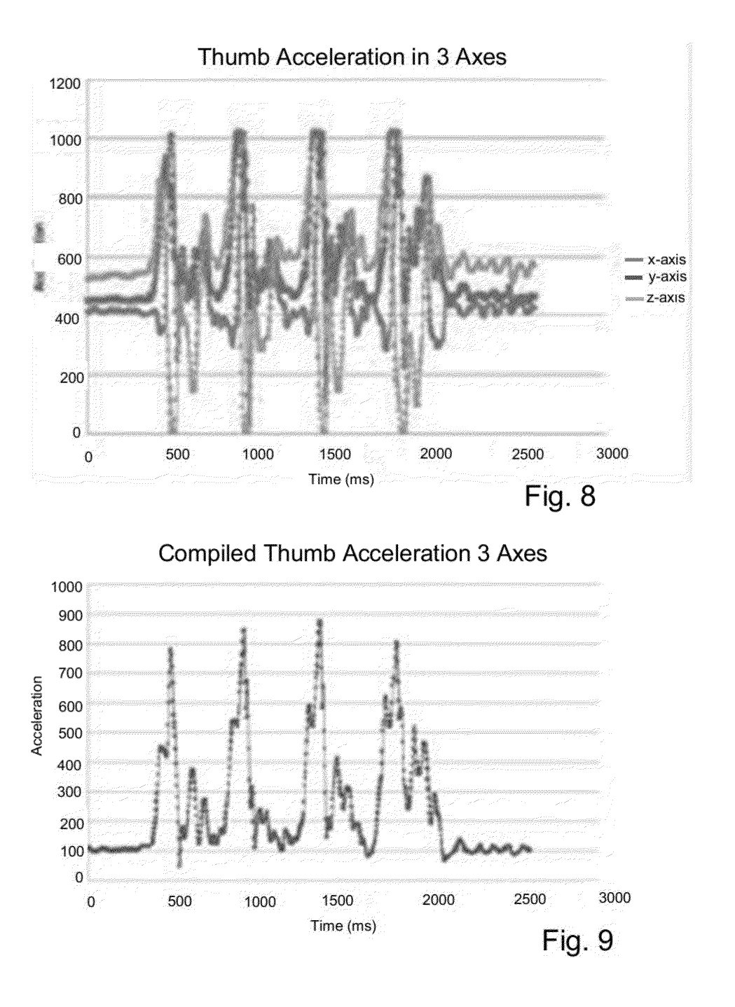

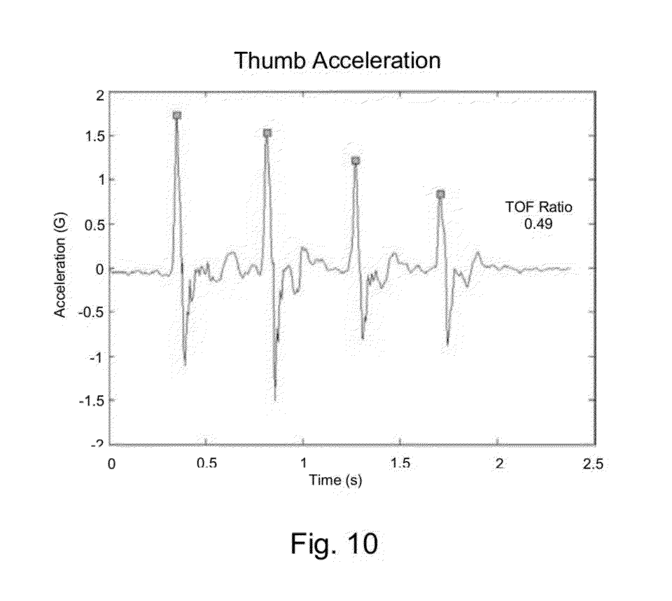

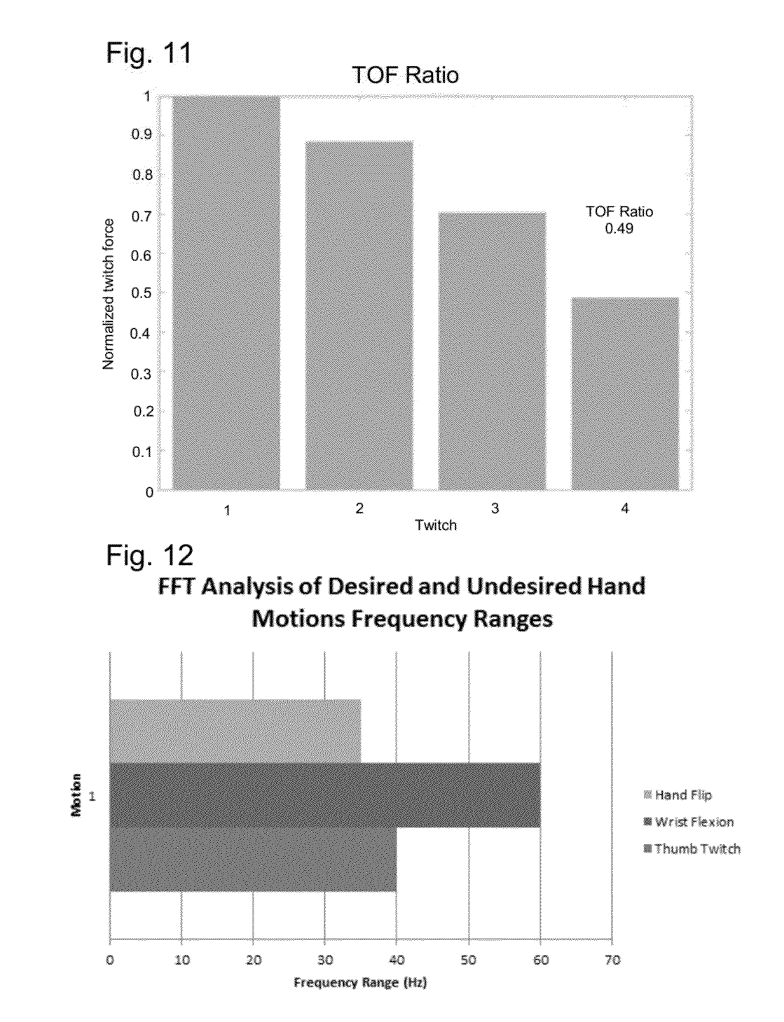

The circuitry and the algorithm that finds relevant data points from acceleration data and presents this data in a meaningful way to the user is described by the flowcharts in FIGS. 6 and 7. Each of the 3 axes has a different baseline based on the device's orientation as illustrated in FIG. 8. The root mean square composite of the 3 axes creates a consistent baseline regardless of clip orientation as illustrated in FIG. 9. The root mean square of 3 axes thereby eliminates the effect of gravity. The software further identifies the peaks belonging to twitches as illustrated in FIG. 10. FIG. 11 illustrates that the observed TOF data was substantially similar to the measured values as shown in FIG. 10. The software could also be utilized to collect and analyze data on devices including but not limited to a mobile device, computer and tablet.

The TOF clip 10 can preferably work with all existing stimulators and the software allows for slight differences in stimulator frequency. Interfaced systems only monitor neuromuscular blockade levels at discrete times throughout surgery whereas the TOF clip 10 continuously monitors muscle movement. This feature allows doctors to be aware of any voluntary movement during surgery. This information may be used to help assess levels of NMB. If there is any movement when the patient should be paralyzed, the algorithm will provide visual and audible warnings so the doctors can ensure the patient's safety. The TOF clip 10 is anesthesiologists' only solution for obtaining objective monitoring while continuing to use their existing independent stimulators.

A Circuitry Design Test was conducted to determine if an analog filter could be designed to remove noise generated from movement not considered a thumb twitch. The test consisted of performing trials of three different hand motions: thumb twitch, wrist flexion and hand flip. The thumb twitch was voluntarily simulated and performed at 2 Hz frequency. The wrist flexion and hand flip motions were performed at random frequencies to simulate random hand movements during surgery. The analysis of the results from this test showed that the frequencies of all three motions were similar, and thus an analog filter may not be appropriate to attenuate the undesired frequencies without also attenuating the thumb twitch signal. See FIG. 12 for supporting data.

The final test is the Peakfinder software Twitch Detection Test. It consists of evaluating the TOF ratio outputted by the code and comparing the value to the observed TOF ratio on the raw acceleration data graph. The observed TOF ratio is calculated by visually determining data points belonging to peaks produced by thumb twitches and using the data points to calculate the TOF ratio. This test has not been performed yet.

To test that the TOF software will work in conditions that may be encountered during surgery, four different tests were performed. This was the key test of AcceleroMetrix's prototype as the unique nature of the device is the software's ability to monitor muscle twitches independently of a nerve stimulator under various conditions. Testing was performed without the use of drugs or nerve stimulation.

All fingers except the thumb were secured to a table. The subject performed four thumb twitches of equal forces. Next, all fingers except the thumb were secured to a table and the subject performed four thumb twitches of decreasing force. The subject then rotated their wrist in pronation and supination motion and performed four thumb twitches of equal force. The subject also moved their wrist in flexion and extension motion and performed four thumb twitches of equal force.

Five subjects completed these tests and each subject performed six trials. The subjects performed the thumb twitches at a frequency of 1 Hz. A metronome was used to help the subject twitch at the correct time. A TOF clip 10 in accordance with an exemplary embodiment was placed on the subject's right thumb and the TOF software graphed the recorded accelerometer signal and calculated the TOF ratio.

The TOF software would pass this test if its calculated TOF ratio was statistically equivalent to the graphically observed TOF ratio. TOF ratio was chosen as the measurement for pass/fail criteria as it is the standard for evaluating NMB. The two TOF ratios were assessed with an equivalence test for means with an equivalence margin of ±0.05. This value was chosen because literature varies on the required TOF ratio to prevent residual paralysis (0.90-1.00). With an equivalence margin of ±0.05 and a product recommendation to reach a TOF ratio of 0.95 before extubation, the software's calculated value would be within the literature values.

The anticipated results of this study are that the TOF ratio calculated by the TOF software will be deemed statistically equivalent to the graphically observed TOF ratio. There are three reasons why these results are anticipated. Firstly, it is simple to code software to identify local signal peaks. Secondly, prior to the execution of this study, AcceleroMetrix completed a study where the desired and undesirable frequencies of the accelerometer signal were identified. The results of this study were used to remove the undesirable portions of the signal to reduce noise. Finally, as the frequency of thumb twitches is consistent in a TOF test, identifying the resulting peaks will be simplified.

The success of the TOF software demonstrates that the TOF clip 10 can be used without being interfaced with a stimulator, decreasing the cost of the device and allowing any existing nerve stimulator to be used with the product.

Possible future uses include:

A research tool to collect data which could provide the basis for new and improved evaluation methods. One example is analyzing the deceleration or rebound of the thumb in arthritic patients to better evaluate neuromuscular blockade in these patients.

Assessment of muscular hand injuries to determine progress of rehabilitation by measuring the force and acceleration of hand movements and application where the measurement force/acceleration of human digits in single or multiple planes may be required.

Application where the measurement of force/acceleration of a human digits in multiple or single planes may be required.

Appropriately modified software can be made compatible with other nerve stimulation tests including but not limited single twitch, tetanus, double burst and post-tetanic count stimulation patterns.

The device can wirelessly transmit data to devices including but not limited to a mobile device, computer or tablet and used to monitor tremors due to disease (e.g. Parkinson's)

Monitor Rehabilitation Progress for Arthritis and Similar Medical Conditions

Educational Drumming Aid

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A monitoring apparatus comprising:
a clip assembly configured to be secured on a patient's digit, the clip assembly housing an accelerometer which measures acceleration in three dimensions;
a microcontroller configured to receive acceleration data from the accelerometer representative of the acceleration measured in each of the three dimensions; and
a processor configured to analyze the acceleration data and determine and output a train-of-four ratio based the acceleration measured in each of the three dimensions.

2. The monitoring apparatus according to claim 1 wherein the microcontroller is configured to filter out unwanted frequencies.

3. The monitoring apparatus according to claim 1 wherein the microcontroller is configured to digitize the acceleration data.

4. The monitoring apparatus according to claim 1 wherein the processor is integral with the microprocessor.

5. The monitoring apparatus according to claim 1 wherein the processor is part of a computing device independent of the microcontroller.

6. The monitoring apparatus according to claim 1 wherein the determined train-of-four ratio is output to and displayed on a display.

7. The monitoring apparatus according to claim 6 wherein the display is selected from one of a monitor, computer screen, tablet and smartphone.

8. The monitoring apparatus according to claim 6 wherein the processor communicates with the display via a wireless transmission.

9. The monitoring apparatus according to claim 6 wherein the processor communicates with the display via a two-way wired or wireless transmission.

10. The monitoring apparatus according to claim 6 wherein the display is a screen integral with the clip assembly.

11. The monitoring apparatus according to claim 1 wherein the processor is configured to compare the measured acceleration data with a baseline value for each of the three dimensions.

12. The monitoring apparatus according to claim 11 wherein the processor is configured to determine a root mean square composite baseline based on the baseline value for each of the three dimensions.

13. The monitoring apparatus according to claim 1 wherein the processor is configured to determine a root mean square composite of the measured acceleration data from the three dimensions and determine a digit twitch has occurred when the composite of the measured acceleration is at a peak compared to the composite baseline.

14. The monitoring apparatus according to claim 1 wherein the clip assembly includes opposed shell members which define a digit opening therebetween and which are biased toward one another to secure the assembly upon a digit positioned within the digit opening.

15. The monitoring apparatus according to claim 1 wherein the processor is configured to continuously determine and output the train-of-four ratio independent of any nerve block stimulation to the patient.

16. A method of monitoring the train-of-four ratio of a patient comprising the steps of:
monitoring acceleration of a digit of the patient utiling an accelerometer which measures acceleration in three dimensions;
transmitting acceleration data from the accelerometer representative of the acceleration monitored in each of the three dimensions:
processing the acceleration data;
determining a train-of-four ratio based the acceleration measured in each of the three dimensions; and
outputting the determined train-of-four ratio on a display.

17. The method according to claim 16, further comprising the step of to filtering out unwanted frequencies.

18. The method according to claim 16, wherein the step of processing the acceleration data includes determining a root mean square composite baseline based on a baseline value for each of the three dimensions and determining a root mean square composite of the monitored acceleration data from the three dimensions.

19. The method according to claim 18 wherein step of determining a train-of-four ratio includes determining that a digit twitch has occurred when the composite of the measured acceleration is at a peak compared to the composite baseline.

20. The method according to claim 16 wherein the acceleration is continuously monitored and the train-of-four ratio is determined and output independent of any nerve block stimulation to the patient.
